(19) United States
(12) Patent Application Publication
(10) Pub. No.: US 2009/0306782 A1
(43) Pub. Date: Dec. 10, 2009

(54) Reverse endoprosthesis for a shoulder joint

(75) Inventor: Hans-Kaspar Schwyzer, Zurich (CH)

Correspondence Address: KNOBBE MARTENS OLSON & BEAR LLP, 2040 MAIN STREET, FOURTEENTH FLOOR, IRVINE, CA, 92614, US

(73) Assignee: Smith and Nephew Orthopaedics AG, Rotkreuz (CH)

(21) Appl. No.: 12/090,231

(22) PCT Filed: Oct. 3, 2006
(86) PCT No.: PCT/CH06/00540

§ 371 Date: Sep. 24, 2008

(30) Foreign Application Priority Data
Oct. 13, 2005 (CH) 1659/05

(51) Int. Cl. A61F 2/40

(52) U.S. Cl. 623/19.12

(57) ABSTRACT

A reverse shoulder joint prosthesis has at least one stemless joint cup or shell (9, 11). The humeral implant can be of at least two-part construction, having a cup or shell (9) and a slide component (11) which has been inserted in the shell or cup or with which the cup or shell has been coated.

The present invention relates to a shoulder joint prosthesis in accordance with the preamble of claim 1.

Joint replacement for the shoulder joint nowadays involves replacement either of only the humerus-side joint face, the humeral head, (hemiarthroplasty) or of both joint faces, that is to say that of the humeral head and that of the glenoid (total arthroplasty).

Both monobloc and modular endoprostheses are used in such procedures. Both the humerus-side implants and those in the scapula are installed both without cement and with cement.

Cemented glenoid implants have a convex rear surface having either so-called pegs or keels for anchoring in the bone. Pegs are pins of more or less round shape which are anchored in bores in the shoulder blade, while so-called keels are keel-shaped structures which are generally mounted centrally on the rear side of the cup in order that the latter can then be anchored in the shoulder blade. Cementless constructions have a sandwich structure which usually consists of a metal part and a polyethylene part anchored therein. The metal part is anchored in the bone by screws, expansion screws, coated pins or expansible pins.

The implants used on the humerus side are for the most part anchored in the bone by means of bone cement. Use is increasingly also being made of cementless systems. Such systems are either manufactured from titanium alloys, which are already biocompatible per se and allow growth of bone thereon, or CoCr alloys or stainless steels are used which have a coating (usually hydroxyapatite) intended to promote bone growth.

Such components are usually made of metal, a cobalt-chromium or titanium alloy or pure titanium, but ceramics and plastics materials are also used.

As slide partner for the ball head, which usually consists of CoCr alloys or ceramics, polyethylene is frequently employed on the scapula side.

The systems described above serve on the one hand to relieve pain and on the other hand, coupled with the relief of pain, to improve mobility. Their use is indicated in the case of wear to the articular cartilage and also in cases of necrosis of the humeral head after fractures.

When, however, mobility is reduced as a result of progressive rotator cuff insufficiency and at the same time the shoulder joint is becoming unstable, so-called reverse prostheses are used. They prevent cranial displacement of the unstable shoulder joint and, by shifting the centre of rotation, provide support for the insufficient muscles and thus an improvement in mobility.

In reverse prostheses, the biomechanics are reversed and a humeral head is mounted glenoid-side on the scapula and a cup is implanted in the humerus. Both cemented and cementless systems are used on the humerus side.

The known reverse systems have the following disadvantages:
1. All commercially available reverse shoulder systems have on the humerus side a metal component in which a polyethylene cup insert is anchored. That metal component, referred to as a stem hereinbelow, is available either in monobloc or modular form. Modular systems allow somewhat better adaptation to the anatomy. In particular, the proximal component needs a very large amount of space in the humerus and requires extensive removal of bone. The distal stem is used for anchoring and, starting from a relatively thin distal round cross-section, widens trumpet-like towards the proximal. In that case too, it is necessary to sacrifice a relatively large amount of bone substance. If the use of a reverse prosthesis later proves to be unbeneficial or if it has to be revised as a result of an infection, only very few bony structures will be available for subsequent implants. In the case of revision, anchoring proves to be quite difficult. An implant anchoring that preserves bone substance as much as possible would be desirable.
2. The angle of inclination assumed by the cup in the humerus largely determines the stability of the shoulder and also has an effect on mobility (range of motion) as well as on any unintentional impingement in the distal scapula region (notching). All the systems currently on the market have a fixed inclination. This can be a disadvantage, depending upon the anatomical characteristics of the patient. It would be desirable for the surgeon to be able to influence the inclination intra-operatively.
3. The distal stem portion of the prosthesis effects the force transmission inter alia in zones which may or may not be so heavily loaded under normal conditions. This can result, especially at the end of the stem, in bone breakdown processes as a result of overloading (stress shielding) and as a consequence lead to fractures. It would be desirable here to have force transmission like that provided by healthy anatomy and produced by biomechanically similar loads.

It is accordingly an aim of the present invention to cause a minimum of damage to the humeral bone by the use of an implant that requires only as much space as is absolutely necessary. When, by virtue of the reverse design, the humerus assumes a cup-like function, the design of the implant should be oriented to that function.

In order to achieve a flexible angle of inclination, that angle should not be predetermined by the implant but rather should be freely selectable by the surgeon intra-operatively.

Load transmission should be effected at the place where it also mainly occurs in healthy bone. That is to say, proximally. Distal stress peaks and inhomogeneities in the force characteristics should be avoided as far as possible.

For solving the problem there is accordingly proposed a shoulder joint prosthesis in accordance with the wording of claim 1.

A reverse shoulder joint prosthesis or implant is proposed which is based on the concept of reverse shoulder endoprosthetics but which, compared with conventional systems, does not have a distal component, that is to say the shell or cup is anchored stemlessly in the proximal humerus. The humerus-side shoulder implant either can consist of at least two components in the modular case or, in an exceptional case, can consist of a single, for example cemented, component.

In accordance with a variant, the humerus-side shoulder implant consists of the said shell or cup as well as a slide component, which is inserted in the shell or with which the inner surface of the shell or cup has been coated. The said shell can be fixed in the humerus either with or without cement. In the cementless configuration, fixation can be effected either by a press-fit or by a screw-like shaping of the outer contour. In the case of a press-fit procedure, the contour of the outer surface of the cup is milled into the bone with slight undersizing relative to the implant. The implant is driven into the resulting hemispherical cavity, the implant likewise being hemispherical, and acquires its primary stability as a result of the so-called press-fit.

When the shell is installed without cement, for better bone integration the shell can be coated with hydroxyapatite or other calcium phosphates and/or osteoinductive materials and also with growth factors. Calcium phosphates are used for better bony integration. There are various sub-groups of calcium phosphates, such as β-tri-calcium phosphate, tetracalcium phosphate, hydroxyapatite, etc., which, depending upon their stoichiometric ratio, are to a greater or lesser degree similar to the calcium phosphate of the bone. In addition to their being used as a direct bone replacement, they are also used as coatings for orthopaedic implants in order to bring about faster and more stable bonding with the bone.

In the case of a press-fit connection, the outer surface of the shell or cup can correspond to a spherical or ellipsoidal surface, but it can also be the surface of a truncated cone. Additional surface shaping, such as flutes, grooves, fins, pins, bone screws or the like, for better fixation of the press-fit connection are likewise possible.

Any through-bores for the introduction of bone screws can be provided with a thread in order to achieve a connection having angular stability.

In the case of a threaded shell, the surface preferably has the geometry of the surface of a truncated cone, but it may also be biconical or spherical.

Materials that come into consideration for the shell or cup when a cementless version is used are especially titanium, titanium alloys, cobalt-chromium alloys (optionally coated), stainless steels (optionally coated), various polymers, such as, especially, PEEK (optionally coated) or CF/PEEK (optionally coated). CF/PEEK denotes carbon-fibre-reinforced polyether ether ketone, the latter being a semi-crystalline thermoplastic material which has long been known in medical technology and is considered to be biocompatible. The coating can consist of either titanium or titanium/HA (hydroxyapatite) layers, which are applied, for example, by means of vacuum plasma spraying or in a deposition process, such as, for example, sol-gel coatings. In addition, corundum-blasted titanium surfaces are especially advantageous for cementless anchoring.

As materials for the shell or cup it is especially possible to use in the cemented version CoCr alloys, stainless steels, PEEK, CF/PEEK. The inlay as slide partner can consist, for example, of polyethylene, ceramics, metal or PEEK, the inlay having, for example, round or oval geometry. A special configuration of the inlay is to be provided towards the scapula to avoid impingement thereon.

The inlay can project beyond the rim of the shell or can be flush therewith or can even fall short of the rim.

As glenoid-side slide partner there can be used either ceramics, plastics material or metal, while on the scapula side it is preferable to use ball segments made of polished metal, such as, for example, CoCr-XX alloys or ceramics.

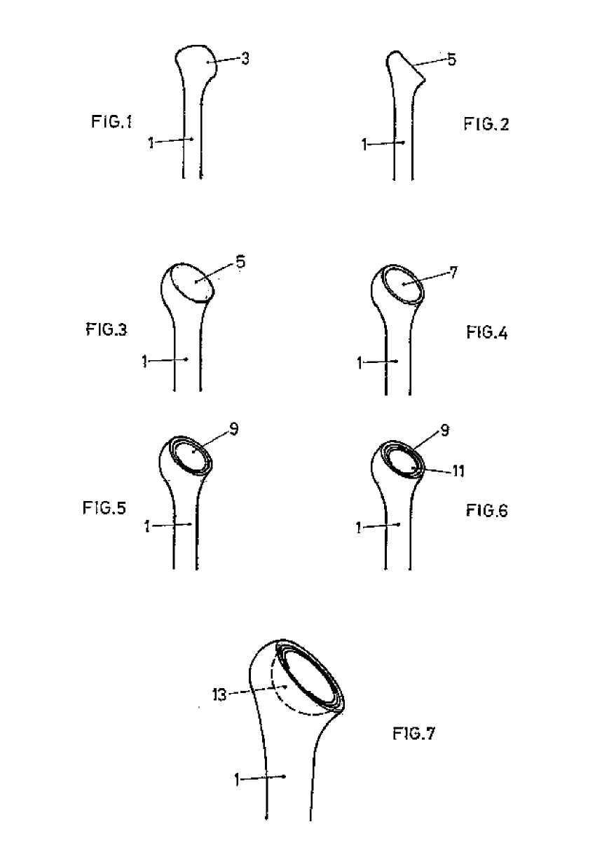

With reference to the FIGS. 1 to 7 described below, implantation of the shell or cup after resection of the humeral head will be described by way of example and diagrammatically, the implantation being carried out analogously to that in the case of hip socket systems.

FIG. 1 shows the humeral bone 1 having the terminally arranged humeral head 3, which may, for example, have become worn or damaged.

FIG. 2 again shows the humeral bone 1 after resection of the humeral head 3, with the result that a largely flat face 5 is formed, directed towards the scapula and provided for installation of a shell or cup, that is to say the humeral implant.

FIG. 3 shows the humeral bone 1 in a perspective side view, having the terminal flat face 5.

For the installation of the shell or cup, first of all a corresponding depression is formed terminally in the humeral bone 1 which is indicated diagrammatically in FIG. 4 by reference numeral 7.

Depending upon the way in which the humeral implant is constructed, that is to say in one, two or three parts, the shell or cup is now inserted into the recess 7. According to FIG. 5, first of all the shell or cup 9 is inserted into the recess 7, and then, as shown diagrammatically in FIG. 6, the slide component 11 is inserted into the shell or cup 9. The shell or cup can be arranged cementlessly in the recess 7, for example by being screwed in, with the outer surface of the shell having screw-like shaping, or by the use of additional bone screws, grooves and the like. Fixation by means of a press-fit connection is likewise possible.

FIG. 7 finally shows the installed humeral implant 13, terminally in the humeral bone 1 in a diagrammatic sectional view, that is to say analogously to a 3D view.

The implantation of the humeral implant according to FIGS. 1 to 7 represents only one possible exemplary example and it will be understood that other installation variants are possible. As already mentioned above, both cementless and cemented implantation can be carried out. It is important to the invention that the humeral implant is of stemless construction in order to avoid the disadvantages of reverse shoulder prostheses mentioned at the beginning.

1-13. (canceled)

14. A shoulder joint prosthesis, said prosthesis being of a reverse construction and comprising at least one stemless joint cup configured for fixation to a humerus.

15. The prosthesis of claim 14, wherein the prosthesis is a humeral implant of at least two-part construction comprising said joint cup and a slide component configured for insertion into said joint cup.

16. The prosthesis of claim 14, wherein the prosthesis is a humeral implant of at least two-part construction wherein said joint cup is coated with a slide component.

17. The prosthesis of claim 14, wherein the joint cup comprises an outer contour with a shape selected from the group consisting of screw-like shaping, and screw-like shaping suitable for cementless fixation of said joint cup to the humerus.

18. The prosthesis of claim 14, wherein an outer surface of the joint cup comprises a coating of a material selected from the group consisting of hydroxyapatite, other calcium phosphates, osteo-inductive materials, growth factors, and any two or more thereof, said coating configured to facilitate integration of the joint cup in the humerus.

19. The prosthesis of claim 14, wherein the construction of an outer surface of the joint cup generally corresponds to a shape selected from the group consisting of a spherical surface, an ellipsoidal surface, and a truncated cone surface.

20. The prosthesis of claim 14, wherein an outer surface of the joint cup comprises at least one element selected from the group consisting of flutes, grooves, fins, pins, and bone screws and the like.

21. The prosthesis of claim 14, further comprising through-bores in the joint cup for the introduction of bone screws configured to facilitate fixation of the joint cup to a humerus bone.

22. The prosthesis of claim 14, wherein the joint cup comprises a material selected from the group consisting of titanium, titanium alloys, CoCr alloys, coated CoCr alloys, stainless steels, coated stainless steels, polymers, coated polymers, polyetheretherketone, coated polyetheretherketone, carbon-fiber-reinforced polyetheretherketone, and coated carbon-fiber-reinforced polyetheretherketone.

23. The prosthesis of claim 22, wherein said joint cup material comprises a coating, said coating comprising a layer selected from the group consisting of a titanium layer, a titanium/hydroxyapatite layer, a titanium layer applied by means of vacuum plasma spraying, a titanium/hydroxyapatite layer applied by means of vacuum plasma spraying, a titanium layer applied by sol-gel or other deposition process, and a titanium/hydroxyapatite layer applied by sol-gel or other deposition process.

24. The prosthesis of claim 14, wherein an outer surface of the joint cup comprises a corundum-blasted titanium surface.

25. The prosthesis of claim 14, wherein the joint cup comprises a material selected from the group consisting of a CoCr alloy, a stainless steel, polyetheretherketone, and carbon-fiber-reinforced polyetheretherketone.

26. Prosthesis according to claim 15, wherein at least one of the joint cup and the slide component comprises a material selected from the group consisting of polyethylene, ceramics, metal, and polyetheretherketone, an inlay of at least one of the joint cup and slide component comprising a geometry selected from the group consisting of round geometry, and oval geometry.

27. The prosthesis of claim 14, further comprising a side ball segment attached to a scapula, the ball segment being movably coupleable to the joint shell and comprising a material selected from the group consisting of polished metal, cobalt-chromium-XX polished metal alloys, and ceramics.

28. A method of providing a shoulder joint prosthesis comprising:
creating a depression in a proximal end of a humerus;
inserting a prosthesis with an outer contour substantially matching a contour of said depression into said depression,
wherein an inner contour of the prosthesis is configured to interface with a corresponding ball segment implanted in a scapula of the shoulder joint.

29. The method of claim 28, wherein the prosthesis is a stemless joint cup.

30. The method of claim 28, wherein the step of inserting the prosthesis into the depression comprises press fitting the prosthesis into the depression in the humerus.

31. The method of claim 28, wherein the step of inserting the prosthesis into the depression comprises screwing the prosthesis into the depression in the humerus.

32. The method of claim 28, further comprising the step of inserting a slide component into said prosthesis, said slide component comprising an outer contour generally matching the inner contour of the prosthesis and configured to facilitate sliding between the and ball segment.

33. The method of claim 28, further comprising the step of choosing an angle of inclination for the depression prior to creating the depression and resecting the proximal end of the humerus to form a surface having said angle of inclination.
